(19) United States
(12) Patent Application Publication
(10) Pub. No.: US 2011/0144694 A1
(43) Pub. Date: Jun. 16, 2011

(54) BONE FIXATION ASSEMBLY

(76) Inventors: Bruno Laeng, Langendorf (CH); Laura Villiger, Langendorf (CH)

(21) Appl. No.: 12/963,858

(22) Filed: Dec. 9, 2010

Related U.S. Application Data
(60) Provisional application No. 61/285,728, filed on Dec. 11, 2009.

(51) Int. Cl. A61B 17/70; A61B 17/56

(52) U.S. Cl. 606/263; 606/277; 606/86 A

(57) ABSTRACT

A bone fixation element includes a bone fixation member having a base and a pair of arms elastically spreadable for reversibly clipping on a bone or an implant, and a connector assembly configured to be attached to the base so as to rigidly fix the fixation member to a bone fixation rod.

### CROSS-REFERENCE TO RELATED APPLICATIONS

This claims the benefit of U.S. Patent Application Ser. No. 61/285,728, filed Dec. 11, 2009, the disclosure of which is hereby incorporated by reference as if set forth in its entirety herein.

### FIELD OF THE DISCLOSURE

The present disclosure generally relates to bone fixation assembly, and in particular to a bone fixation assembly configured to fix a bone fixation rod between two or more vertebrae, in order to stabilize a part of the spinal cord.

### BACKGROUND

Conventional bone fixation elements can be provided as pedicle screws that screw in to the pedicles of vertebral boies. The pedicle screws can include a bone anchor retained within an anchor seat and captured by a collet. Pedicle screw assemblies include a plurality of pedicle screws joined by a bone fixation rod that extends through rod slots formed in the pedicle screws. It has been found that conventional pedicle screws can be oversized for the cervical vertebrae.

### SUMMARY

In accordance with one embodiment, bone fixation assembly includes a bone fixation element having a bone fixation member that is attachable to a bone, in particular to a lamina of a vertebra without affecting the bone or lamina of a vertebra and which is rigidly connectable to a bone fixation rod such that the rod is aligned in the direction of the spinal cord, and a connector assembly that is configured to connect the bone fixation member to the bone fixation rod. For instance, the connector assembly can be configured to releasably fix the bone fixation rod to the bone fixation member. The connector assembly can be polyaxially pivotably linked to the bone fixation member, or can be configured as a cable tie.

The bone fixation member can include a fixation body that includes a base and a pair of laterally spaced arms that extend from the base. The arms can be elastically spreadable, and configured to reversibly clamp onto a lamina of a vertebra. The bone fixation member can further include a connection interface attached to the fixation body, for instance at the base, and is configured to attach to the connector assembly so as to rigidly fix the bone fixation member the bone fixation rod.

In accordance with one embodiment, the bone fixation member can be made from a material with a Young's modulus greater than 30 GPa, preferably greater than 100 GPa. Suitable materials for the bone fixation member are metallic materials as stainless steel, cobalt-chromium alloys, e.g. Co28Cr6Mo with a Young's modulus of 241 GPa, and titanium alloys, e.g. TiNbTaZr with a Young's modulus of 30-100 GPa, Ti15Mo α+β with a Young's modulus of 105 GPa and Ti15Mo β with a Young's modulus of 78 GPa.

In another embodiment, the bone fixation member is made from a memory metal, preferably Nitinol with a Young's modulus of 30-75 GPa.

In a further embodiment the bone fixation member is made from reinforced PEEK with a Young's modulus of >30 GPA.

In another embodiment the bone fixation member has a closable form for enclosing the entire periphery of a longitudinal rod. Due to this configuration a longitudinal rod can be rigidly fixed to the bone fixation member. The longitudinal rod is secured against an unintended removal from the bone fixation member.

In a further embodiment the bone fixation member can be secured to a longitudinal rod with respect to relative translational and rotational movement.

In yet another embodiment the bone fixation member and the connector assembly are rigidly connected to each other. Therewith the advantage can be achieved that the longitudinal rod is securely retained in its position aligned with the spinal cord.

In a further embodiment the bone fixation member oriented such that the fixation rod extends substantially in the plane defined by the fixation body when the fixation rod is fixed to the fixation member.

In another embodiment of the bone fixation member the fixation member is formed as a cable tie. This configuration allows a configuration of the bone fixation member with a small volume of the fixation member.

In another embodiment of the bone fixation member the fixation member comprises a head fixedly arranged at the outer part of the base and a connector assembly linked to the head by a releasably fixable polyaxially pivotable joint and allowing to releasably fix a longitudinal rod to the bone fixation member. This configuration allows the advantage that the longitudinal rod must not necessarily be adapted to the position of each of the bone fixation members along the spinal column. The polyaxially pivotable joint offers the possibility to adjust the fixation member with regard to the position of longitudinal rod.

In a further embodiment, the fixation body of the bone fixation member substantially defines an elliptical C-shape having a first dimension and a second dimension that is less than the first dimension. The substantial arc of the fixation body can extend along a distance within a range that is greater than approximately 240°, for instance greater than approximately 260°, and can be less than approximately 290°, for instance less than approximately 280° measured on a circumcircle of the arc. In accordance with one embodiment, the substantial arc of the fixation body can extend along a distance that is approximately 270° measured on the circumcircle of the arc.

In yet another embodiment the bone fixation member includes more than two arms, preferably four arms that can be arranged in an X-shape or any alternative shape, and can form a passage with an ellipse-like or elliptical C-form or any alternative form as desired.

In another embodiment the base of the fixation body of the bone fixation member has an inner surface the defines a first radius of curvature, and at least one or both of the arms has an inner surface that defines a second radius of curvature that is less than the first radius of curvature.

In another embodiment of the bone fixation member each of the two arms has a free end opposite the base so that between the two free ends an opening remains with a first initial width measured in an unloaded or unflexed state, and can be expanded by an expansion force so as to define a second flexed width that is greater than the first initial width. When the expansion force is released, the fixation body can be firmly fixed on the lamina. The first and second widths can be dimensioned as follows, in accordance with various embodiments.

The first initial width can be within a range that is greater than approximately 12 mm, for instance greater than approximately 13 mm, and less than approximately 16 mm, for instance less than approximately 15 mm. In accordance with one embodiment, the first initial width can be approximately 14 mm. The second flexed width can be within a range that is greater than approximately 16 mm, for instance greater than approximately 18 mm, and less than approximately 22 mm, for instance less than approximately 20 mm. In accordance with one embodiment, the second flexed width can be approximately 19 mm. Thus, the difference between the first initial width and the second flexed width can be within a range that is greater than approximately 3 mm, for instance greater than approximately 4 mm, and less than approximately 7 mm, for instance less than approximately 6 mm. In accordance with one embodiment, the difference between the first initial width and the second flexed width can be approximately 5 mm.

The bone fixation member, for instance the fixation body, can define a relative spreadability that is defined by the ratio of the first initial width/the second flexed width. In accordance with one embodiment, the relative spreadability can be within a range that is greater than approximately 0.6, for instance greater than approximately 0.7, and less than approximately 0.8, for instance less than approximately 0.75. In accordance with one embodiment, the relative spreadability can be approximately 0.74. Otherwise stated, the bone fixation member, for instance the fixation body, can define a ratio of the second flexed width/the first initial width that is within a range greater than approximately 1.25, for instance greater than approximately 1.3, and less than approximately 1.5, for instance less than approximately 1.4. In accordance with one embodiment, the ratio of the second flexed width/the first initial width is approximately 1.36.

the base of the bone fixation member can comprise two recesses arranged symmetrically at equal distances from the short axis in the inner side of the base.

In again a further embodiment of the bone fixation member the each of the two arms comprises an engagement member configured to engage an insertion instrument, preferably in the form of a projection or of a recess.

In another embodiment the arms of the bone fixation member has an inner surface which is coated with hydroxylapatite, a polymer or titanium. The coating allows the advantage that the friction between the arms of the arc-shaped element and the bone can be reduced.

In accordance with another aspect, a method is provided for stabilizing a part of the spinal cord, particularly in the area of the cervical spine by using at least two bone fixation elements comprising the following steps:
a) establishing an incision each for a posterior approach to each vertebral body to be treated;
b) attaching a bone fixation member to the insertion instrument;
c) spreading the bone fixation member by advancing the rod of the insertion instrument;
d) inserting and positioning the bone fixation member over the lamina of a first vertebral body to be treated;
e) releasing the bone fixation member by reversing the rod so that the bone fixation member is clipped over the lamina;
f) repeating steps b) to e) for each vertebral body to be treated;
g) inserting the longitudinal rod into the fixation member, e.g. by snapping a connector assembly onto each of the heads of the bone fixation members attached to the laminae of the vertebral bodies and positioning a longitudinal rod in the channels of the connector assemblies attached to the vertebral bodies, and by mounting an insert screw on each connector assembly and fastening the insert screws; and
h) fixing the longitudinal rod in the fixation member, e.g. by fastening the set screws in each connector assembly in order to fix the longitudinal rod to the bone fixation members.

In accordance with a further aspect, an insertion instrument is provided for clipping a bone fixation member on a lamina of a vertebral body. the insertion instrument comprises:
A) a clamp comprising an engagement member configured to engage the two arms of the bone fixation member; and
B) a shaft with a pusher that is positionable on the top side of the head of the bone fixation member and which can be advanced relative to the clamp in order to spread the two arms of the bone fixation member.

### BRIEF DESCRIPTION OF THE DRAWINGS

Several embodiments will be described in the following by way of example and with reference to the accompanying drawings in which:

FIG. 1 is a perspective view of a bone fixation assembly constructed in accordance with one embodiment including a plurality of bone fixation elements and a bone fixation rod connected between the bone fixation elements, showing the bone fixation assembly implanted onto a spine;

FIG. 2 is a sectional perspective view of one of the bone fixation elements illustrated in FIG. 1, including a connector assembly and a bone fixation member;

FIG. 3 is a top plan view of the bone fixation member illustrated in FIG. 2, including a connection interface and a head that extends from the fixation body;

FIG. 4A is a perspective view of an insertion instrument constructed in accordance with one embodiment, shown coupled to a bone fixation member;

FIG. 4B is an enlarged perspective view of a distal end of the insertion instrument illustrated in FIG. 4A, shown coupled to the bone fixation member;

FIG. 5A is a perspective view of the bone fixation member illustrated in FIG. 3, shown implanted onto a lamina of one vertebral body;

FIG. 5B is a perspective view of the attachment of the connector assembly illustrated in FIG. 2 onto the bone fixation member illustrated in FIG. 5A, along with a second bone fixation member implanted onto a lamina of an adjacent vertebral body;

FIG. 5C is a perspective view of the insertion of a spinal fixation rod into a plurality of connector assembly assemblies;

FIG. 6 is a top plan view of the bone fixation member as illustrated in FIG. 3, but showing the connection interface constructed in accordance with another embodiment;

FIG. 7A is a sectional side elevation view of a portion of a bone fixation assembly including a portion of a bone fixation member a bone fixation rod, and a connector assembly configured to fasten the bone fixation rod to the bone fixation member; and

FIG. 7B is a side elevation view of the portion of the bone fixation assembly illustrated in FIG. 7A.

### DETAILED DESCRIPTION

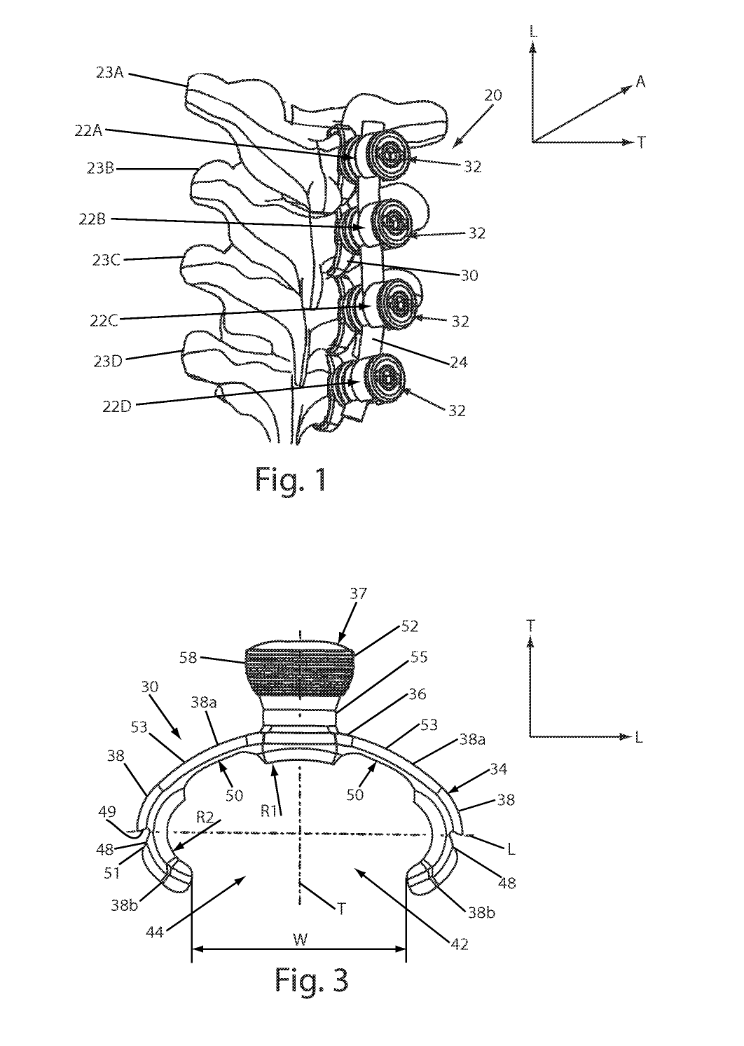

Referring initially to FIG. 1, a bone fixation assembly 20 includes one or more bone fixation elements 1 such as four bone fixation elements 22A-D as illustrated in FIG. 1, connected by a bone fixation rod 24 and implanted onto underlying bone, such as respective vertebrae 23A-D. In accordance with the illustrated embodiment, the bone fixation elements 22A-D are spaced substantially along a longitudinal direction, and the fixation rod 24 is generally elongate along the longitudinal direction L (and thus may be curved and deviate slightly with respect to the longitudinal direction L), such that the fixation rod 24 is substantially aligned in the direction of the spinal cord. Each bone fixation element 22A-D is illustrated as extending from the respective vertebrae 23A-D in a substantially transverse direction T that is substantially perpendicular to the longitudinal direction L, and further defines a lateral direction A that is substantially vertical with respect to the transverse direction T and the longitudinal direction L.

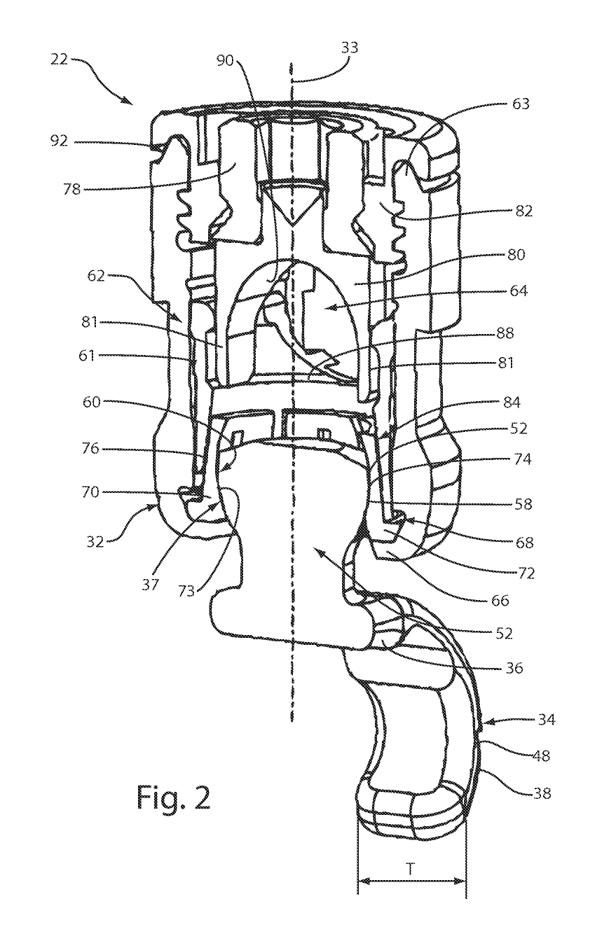

Unless otherwise specified herein, the terms “lateral,” “longitudinal,” and “transverse” are used to describe the directional components of the bone fixation assembly 20. In the orientation illustrated in FIG. 2, each bone fixation element 22 extends vertically along the transverse direction T, and horizontally along the lateral direction A and longitudinal direction L. Thus, it should be appreciated that while the longitudinal and lateral directions are illustrated as extending along a horizontal plane, and that the transverse direction is illustrated as extending along a vertical plane, the planes that encompass the various directions may differ during use. For instance, when the bone fixation elements 22A-D are implanted onto a spine, the longitudinal direction L extends generally along the superior-inferior (or cranial-caudal) direction, while the plane defined by the lateral direction A and the transverse direction T lie generally in the anatomical plane defined by the medial-lateral direction, and the anterior-posterior direction, respectively. Accordingly, the directional terms “vertical” and “horizontal” are used to describe the implant 10 and its components as illustrated merely for the purposes of clarity and illustration.

The words “inward,” “outward,” “upper,” “lower,” “distal,” and “proximal,” can refer to directions toward or away from, respectively, the geometric center of the bone fixation assembly 20 and its components. The words, “anterior”, “posterior”, “superior,” “inferior” and related words and/or phrases designate preferred positions and orientations in the human body to which reference is made and are not meant to be limiting. It should further be appreciated that while round structures define diameters as described herein, the round structures could be replaced with alternative (e.g., polygonal) structures which would define alternative cross-sectional dimensions opposed to diameters. The term “diameter” as used herein is intended to include all such alternatives unless otherwise specified. The terminology includes the above-listed words, derivatives thereof and words of similar import.

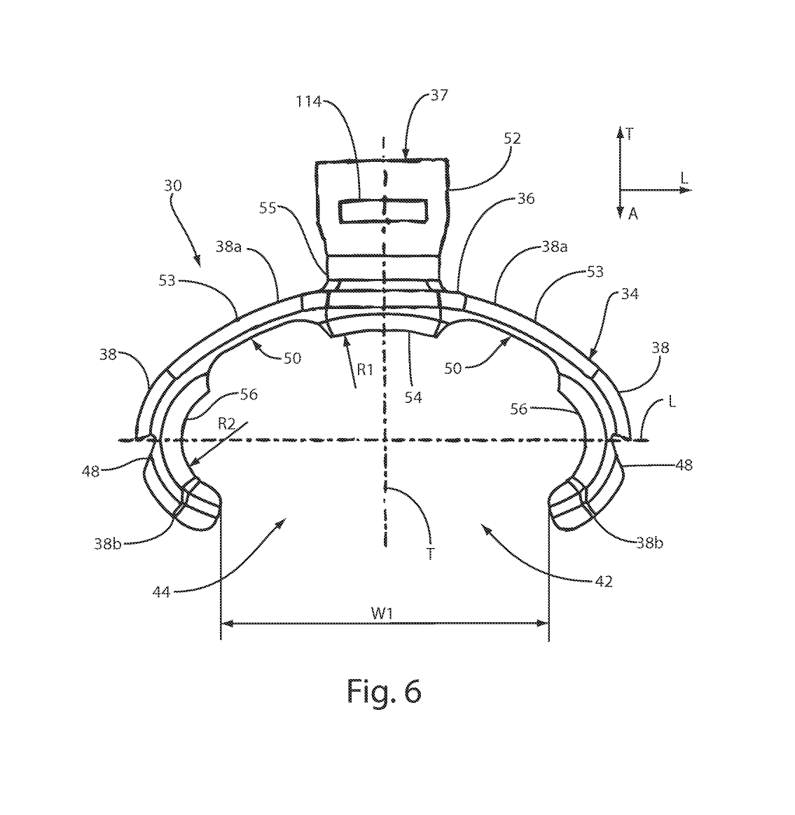

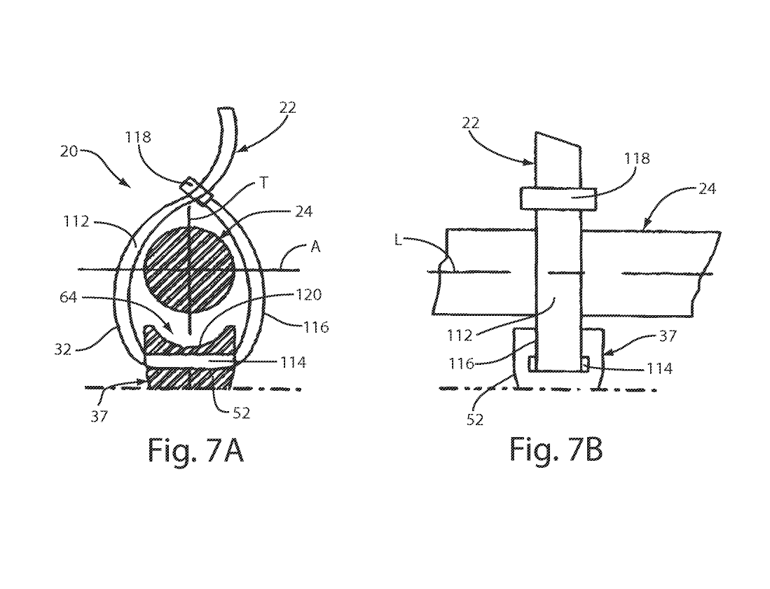

With continuing reference to FIG. 1, each bone fixation element 22A-D includes a bone fixation member 30, which is illustrated as a bone anchor, that is implanted (e.g., clipped) onto a lamina of the corresponding vertebra 27A-D, and a connector assembly 32 that is configured to be connected between the bone fixation members 30 and the fixation rod 24, so as to attach and fix the fixation rod 24 to the bone fixation members 30. The connector assembly 32 can be configured to releasably fix the bone fixation member 30 to the bone fixation rod 24. As will be appreciated from the description below, the connector assembly 32 can be polyaxially pivotably linked to the bone fixation member 30 in a first configuration, and subsequently tightened in a second configuration so as to be fixed to the bone fixation member 30 with respect to relative translational and rotational movement, thereby further fixing the bone fixation member 30 to the fixation rod 24 with respect to relative translational and rotational movement. Alternatively, as is described in more detail below, the connector assembly 32 can be configured as a cable tie (see FIGS. 6-7B).

Unless otherwise specified, the bone fixation assembly 20 and its components can be made from titanium-aluminum-niobium alloy (TAN), implant-grade 316L stainless steel, or any suitable alternative implant-grade material. The bone fixation elements 22A-D can be generally implanted in any region of the spine, for instance at lumbar, thoracic, or cervical regions. In this regard, when the bone fixation elements 22A-D are joined by the fixation rod 24, the bone fixation assembly 20 fixes the relative position of the vertebrae 23A-D. Accordingly, the bone fixation elements 22A-D can be referred to as spine fixation elements, the fixation rod 24 can be referred to as a spinal fixation rod, and the bone fixation assembly 20 can be referred to as a spine fixation assembly. However, it should be appreciated that the bone fixation assembly 20 can also be used for fixation of other parts of the body, such as joints, long bones, or bones in the hands, face, feet, extremities, cranium, and the like. The fixation rod 24 can be substantialy cylindrical or tubular in shape, and can be substantially rigid or flexible as desired. The fixation rod may include, but is not limited to, a solid body, a non-solid body, a flexible or dynamic body, or the like, and can assume any alternative shape as desired.

Referring now to FIG. 3, each bone fixation member 30 can be provided as a bone anchor, having a fixation body 34 configured to be mounted onto an underlying bone, such as a vertebra 23, and a connection interface 37 that extends from the fixation body 34 and is configured to attach to a respective connector assembly 32 so as to rigidly fix the bone fixation member 30 the bone fixation rod 24.

The bone fixation member 30 can be made from any suitable material, for instance a material having a Young's modulus greater than 30 GPa, such as greater than 100 GPa. For instance, the bone fixation member 30 can be made from a metallic material such as stainless steel, cobalt-chromium alloys, e.g. Co28Cr6Mo which can have a Young's modulus of approximately 241 GPa, and titanium alloys, e.g. TiNbTaZr which can have a Young's modulus of between approximately 30 GPa and approximately 100 GPa, Ti15Mo α+β which can have a Young's modulus of approximately 105 GPa, and Ti15Mo β which can have a Young's modulus of approximately 78 GPa. In accordance with another embodiment, the bone fixation member 30 can be made from a shape memory material or metal, such as Nitinol which has a Young's modulus of between approximately 30 GPa and approximately 75 GPa. In accordance with yet another embodiment, the bone fixation member 30 can be made from reinforced PEEK with a Young's modulus greater than approximately 30 GPa.

The fixation body 34 includes a base 36 and a pair of longitudinally spaced arms 38 that extend from laterally opposed sides of the base 36. The arms 38 define respective inner surfaces that face each other, and opposed outer surfaces. The inner surfaces can be coated with hydroxylapatite, a polymer or titanium, so as to reduce friction between the arms 38 and the bone received in the arms 38. The arms 38 can define any suitable size and shape as desired, and are curved in accordance with the illustrated embodiment. For instance, the opposed arms 38 can be substantially concave with respect to each other. Thus, each arm 38 can define a proximal end 38a that curves away from the proximal end 38a of the opposed arm 38, and a free distal end 38b opposite the base that curves toward the free distal end 38b of the opposed arm 38. Thus, the fixation body 34 can be substantially arc-shaped in accordance with the illustrated embodiment, and for instance can define a substantially elliptical C-shape that can define a first longitudinal dimension that extends along a longitudinal axis L, and a second transverse dimension that extends along the transverse axis T and can be smaller than the first longitudinal dimension. The first longitudinal dimension can define the longest longitudinal dimension of the fixation body 34 between the arms 38, and the second transverse dimension can define the longest transverse dimension of the fixation body 34. Thus, the first longitudinal dimension is longer than the second transverse dimension. The base 36 defines an inner radius of curvature R1 and each of the two arms 38 has an inner radius of curvature R2 that is less than R1 as illustrated.

It should be appreciated, however, that the fixation body 34 can define any suitable alternative shape as desired. For instance, the arms 38 can alternatively comprise at least one segment, alone or in combination with at least one curved segment. Accordingly, when the fixation member 30 is in a first initial or unflexed position, the arms 38 can define a first distance that extends between the proximal ends 38a, a second distance that extends between a location between the proximal ends 38a and 38b that extends parallel to the first distance and is greater than the first distance, and a third distance that extends between the distal ends 38b parallel to the first and second distances and is less than the second distance.

In accordance with one embodiment, the fixation body 34 of the bone fixation member 30 can substantially defines the shape of a substantial arc that can extend along a distance within a range that is greater than approximately 240°, for instance greater than approximately 260°, and can be less than approximately 290°, for instance less than approximately 280° measured on a circumcircle of the arc. In accordance with one embodiment, the substantially arc of the fixation body can extend along a distance that is approximately 270° measured on the circumcircle of the arc.

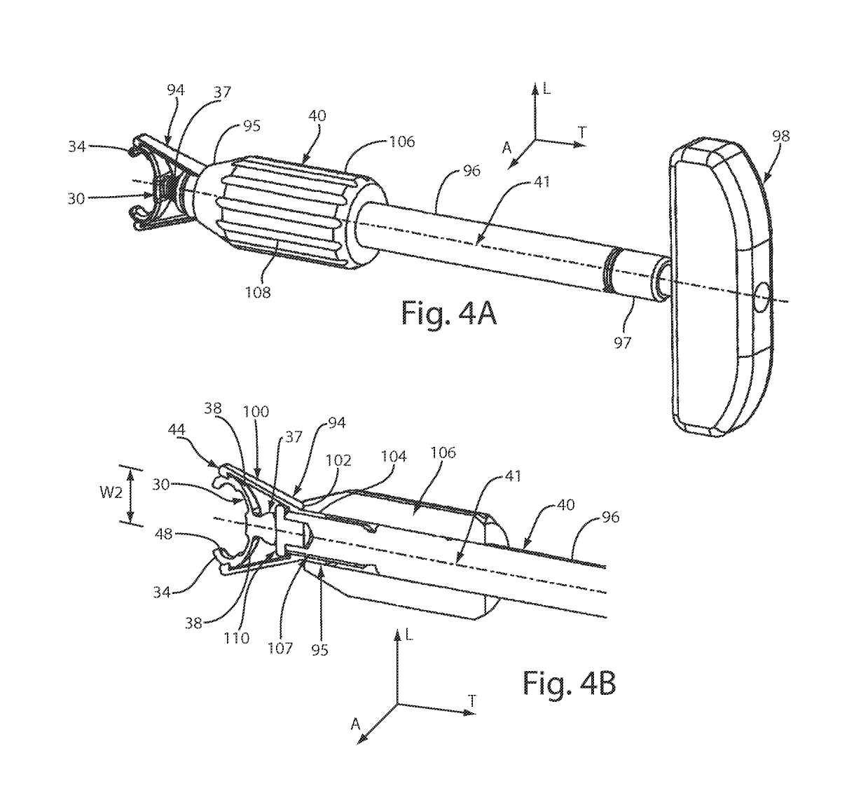

The fixation body 34, and in particular the base 36 and arms 38 defines a bone-receiving space 42 having a mouth 44 that defines a width W between the disposed between the distal ends 38b of the opposed arms 38. The fixation body 34, and in particular the arms 38, can move between a first initial position such that the distal ends 38b of the arms 38 define a first initial width W1 and a second flexed position such that the distal ends 38b of the arms 38 define a second flexed width W2 (see FIG. 4B) that is greater than the first initial width W1. For instance, the arm 38 can be engaged and spread apart by an insertion instrument 40 as described below with reference to FIGS. 4A-B. The initial width W1 can be less than the lamina onto which the fixation member 30 is to be mounted, and the second width can be greater than the lamina onto which the fixation member 30 is to be mounted, such that the arms 38 can be fit over the lamina of the underlying vertebra 23 when a spreading force is applied to the arms 38 that causes the arms 38 to flex away from each other. Accordingly, the bone, such as the lamina, can be inserted through the mouth 44 and into the bone-receiving space 42. When the spreading force is released, the spring force of the arms 38 causes the arms to recoil toward each other and clamp (e.g., be firmly fixed) onto the bone, such as the lamina. Thus, the arms 38 can be elastically spreadable, and configured to reversibly clamp onto a lamina of a vertebra.

The first initial width W1 can be within a range that is greater than approximately 12 mm, for instance greater than approximately 13 mm, and less than approximately 16 mm, for instance less than approximately 15 mm. In accordance with one embodiment, the first initial width W1 can be approximately 14 mm. The second flexed width W2 can be within a range that is greater than approximately 16 mm, for instance greater than approximately 18 mm, and less than approximately 22 mm, for instance less than approximately 20 mm. In accordance with one embodiment, the second flexed width W2 can be approximately 19 mm. Thus, the difference between the first initial width W1 and the second flexed width W2 can be within a range that is greater than approximately 3 mm, for instance greater than approximately 4 mm, and less than approximately 7 mm, for instance less than approximately 6 mm. In accordance with one embodiment, the difference between the first initial width W1 and the second flexed width W2 can be approximately 5 mm.

The bone fixation member 30, for instance the fixation body 34, can define a relative spreadability that is defined by the ratio of the first initial width W1/the second flexed width W2. In accordance with one embodiment, the relative spreadability can be within a range that is greater than approximately 0.6, for instance greater than approximately 0.7, and less than approximately 0.8, for instance less than approximately 0.75. In accordance with one embodiment, the relative spreadability can be approximately 0.74. Otherwise stated, the bone fixation member 30, for instance the fixation body 34, can define a ratio of the second flexed width W2/the first initial width W1 that is within a range greater than approximately 1.25, for instance greater than approximately 1.3, and less than approximately 1.5, for instance less than approximately 1.4. In accordance with one embodiment, the ratio of the second flexed width W2/the first initial width W1 is approximately 1.36.

The fixation body 34 further defines at least one engagement member configured to mate with a complementary engagement member of the insertion tool 40, such that the insertion tool 40 can apply an expansion force to the fixation body 34, and in particular to the arms 38 that causes the arms 38 to flex from their first initial or unflexed positions to their second flexed positions. The engagement member of the fixation body 34 can be configured as desired, such as a projection or a recess, and is illustrated as a recess that forms a groove 48 that extends into each arm and is configured to receive a respective engagement member in the form of an engagement tooth 44 of the insertion instrument 40 (see FIGS. 4A-B). In particular, each groove 48 can extend longitudinally into the outer surface of each of the longitudinally spaced arms 38 at a location between the proximal and distal ends 38a and 38b. In accordance with the illustrated embodiment, the grooves 48 are disposed between the distal ends 38b and location midway between the proximal and distal ends 38a and 38b. Each groove 48 can define a substantially saw-tooth cross-section viewed in the lateral direction, which is orthogonal to the transverse-longitudinal plane defined by the fixation body 24. In particular, each groove 48 can define a steep flank 49 that extends toward the connection interface 37 along a direction from the outer surface of the respective arm 38 toward the inner surface of the respective arm 38. The steep flank 49 can be located on the first longitudinal dimension, which is the longest dimension of the fixation body 34 between the arms 38. Each groove 48 can further define a flat flank 51 that is angled with respect to the steep flank 49. It will be understood from the description below that the steep flank is configured to receive the expansion force from the insertion instrument 40, as applied by the engagement tooth 44.

The fixation body 34 further defines a pair of recesses 50 that extend into the inner surface of the respective pair of arms 38 at a location that can be arranged symmetrically at substantially equal distances from the second transverse dimension of the fixation body 34. Each recess 50 defines a thinned region of the arms 38 that increase the elasticity, or flexibility, of the arms 38. Thus, the arms 38 can define respective hinges 53 at the recesses 50 that facilitate flexing of the arms between the first initial or unflexed position and the second flexed position. Furthermore, the fixation body 34 defines a central section 54 disposed adjacent and proximal to the recesses 50 (e.g., at the base 36) that is thicker than the hinges 53 and defines a rigid anchorage that is configured to connect to the connector assembly 32 and facilitate fixation of the bone fixation member 30 to the fixation rod 24. The fixation body 34 defines a distal section 56 of the arms 38 disposed adjacent and distal to the recesses 50 (e.g., at the base 36) that is thicker than the hinges 53 and defines a rigid anchorage that is configured to connect to a bone (e.g., lamina) that is received in the bone-receiving space 42.

Referring now to FIGS. 2-3, the connection interface 37 of the bone fixation member 30 defines a head 52 that extends from the outer surface of the base 36, for instance via a transverse post 55. The head 52 can be integral with the base 36, or can alternatively be discreetly connected to the base 36. The head 52 can define an outer engagement surface 58 that can be round, e.g., substantially spherical, and is configured to interface with the connector assembly 32 as part of a polyaxial ball-and-socket joint. The head 52 can be coincident with the second transverse dimension of the fixation body 34, and can extend centrally along the second transverse dimension. The connector assembly 32 defines a complementary socket 60 that is configured to receive the ball-shaped head 52 such that the connector assembly 32, and the fixation rod 24 that is received by the connector assembly 32, is polyaxially pivotable with respect to the bone fixation member 30 when the connector assembly 32 is in a first unlocked configuration. Accordingly, the fixation rod 24 can be curved or otherwise extend in a direction slightly offset from the longitudinal direction L, and can thereby follow the contour of the spinal column, while the angular orientation of the fixation member 30 can be adjusted with respect to the fixation rod 24. The connector assembly 32 can subsequently be tightened to rigidly connect the bone fixation member and the connector assembly 32, thereby preventing movement of the fixation rod 24 relative to the bone fixation member 30 and securely retaining the fixation rod 24 in its position aligned with the spinal cord as desired.

The connector assembly 32 can be configured as desired, as is well known in the field of pedicle screws or hooks. For instance, the connector assembly 32 can include a hollow cylindrical sleeve 62 that defines a central bore hole 61 elongate along a central transverse axis 33. The central bore 61 defines an internal channel 64 having an upper (e.g., outer transverse) open end 63 that is configured to receive the fixation rod 24 into the channel 64, for instance before a set screw 78, plug 80, and insert screw 82 are connected, either directly or indirectly, to the sleeve 62 so as to close the open end 63 of the channel 64. If desired, the channel 64 can also be open towards the side or be formed as an oval bore hole. The sleeve 62 defines a lower (e.g., inner transverse) end 66 that defines a recess 68 that can be substantially ring-shaped. The connector assembly 32 includes a fastener such as a spring chuck 70 that is inserted into the lower end 66 of the bore hole 61 of the sleeve 62. The spring chuck 70 includes an outer flange 72 that is inserted into the recess 68, so as to substantially fix the spring chuck 70 to the sleeve 62 with respect to translation along the central axis 33 of the bore hole 61. The outer flange 72 of the spring chuck 70 can be radially displaceable inside the recess 68.

The spring chuck 70 defines an inner engagement surface 73 that defines an internal cavity 74, which can be shaped as a portion of a hollow sphere. Thus, the inner engagement surface 73 can likewise be substantially spherical. The spring chuck 70 further defines a plurality of slots 74 that allow the spring chuck 70 to be substantially homogeneously expanded and compressed as the connector assembly 32 iterates between its first unlocked configuration and its second locked configuration. Because the outer flange 72 of the spring chuck can be radially displaced when connected to the outer sleeve 62, the head 52 of the bone fixation member 30 can be snapped into the spring chuck 70, such that the head is disposed in the internal cavity 74, and can further be snapped out of the spring chuck 70 as desired while the connector assembly 32 is in the first unlocked configuration. When the head 52 is disposed inside the internal cavity 74, the outer engagement surface 58 can abut the complementary inner engagement surface 73 of the spring chuck 70 such that the connector assembly 32 and the fixation member 30 can pivot polyaxially with respect to each other while the connector assembly 32 is in the first unlocked configuration, prior to tightening the set screw 78 which causes the connector assembly 32 to assume the second locked configuration.

The spring chuck 70 further defines an outer surface 76 that tapers conically inward along a direction toward its upper end. The connector assembly 32 further includes a hollow cylindrical insert 84 that is shaped complementarily conical to the outer surface 76 of the spring chuck 70, and can slide down (or transversely inward) within the bore hole 61 at its lower end between the spring chuck 70 and the sleeve 62, thereby providing a radial compressive force onto the outer surface 76 as the conical surfaces ride along each other, thereby causing the inner engagement surface 73 to bear against the outer engagement surface 58 of the head 52. For instance, the set screw 78 can be tightened against the plug 80, which has at least one leg 81 such as a pair of legs 81 that can bear against a shoulder 88 of the insert 84, that causes the insert to translate downward (or transversely inward). Furthermore, the plug 80, which can alternatively be integral with the set screw 78, defines an inner surface 90 that can be substantially spherical so as to tighten the fixation rod 24 that is disposed in the internal channel 64 against the sleeve 62, thereby locking the fixation rod 24 with respect to movement relative to the connector assembly 32, and thus also relative to the bone fixation member 30 when the connector assembly is in its second locked configuration. The bone fixation member 30 can be oriented such that the fixation rod 24 extends substantially in a plane defined by the fixation body 34 when the fixation rod 24 is fixed to the fixation member 30.

As the set screw 78 is tightened, the plug 80 can press the fixation rod 24 onto the insert 84, for instance at the shoulder 88, which can also cause the insert 84 to translate downward. As the insert 84 translates downward, the insert 84 applies a radial compressive force onto the spring chuck 70 that can be sufficient so as to frictionally lock the spring chuck 70 onto the head 52 disposed in the internal cavity 74. The radial compressive force can be released so as to iterate the connector assembly 32 from the second locked configuration to the first unlocked configuration.

The insert screw 82 can essentially alter the inner thread in the sleeve 62 that mates with the set screw 78. It is appreciated that the inner thread is interrupted by the channel 64, and the insert screw 82 can alter the inner thread so as to define a peripherally closed inner thread such that the set screw 78 can advance unhinderedly. Because the bore hole 61 in the sleeve 62 is penetrated by the channel 64 which is open at the upper end 63 of the sleeve 62 and the sleeve 62 can be weakened at this location, the insert screw 82 includes a ring shaped groove 92 that receives the upper end 63 of the sleeve 62 so as to prevent the sleeve 62 from widening as the set screw 78 is tightened.

The connector assembly 32 can also be iterated from the second locked configuration to the first unlocked configuration by loosening the set screw 78, which relieves the pressure of the plug 80 on the shoulder 88 of the insert 84, thereby removing the radial compressive force onto the spring chuck 70.

It should be appreciated that while the connector assembly 32 has been described in accordance with one embodiment, various embodiments of a device that connect a fixation rod with a pedicle screw including a polyaxially pivotable coupling between the device and a pedicle screw are described, for instance in U.S. Pat. No. 6,248,105, the disclosure of which is hereby incorporated by reference as if set forth in its entirety herein.

Referring now to FIGS. 4A-B, an insertion instrument 40 is elongate along a central axis 41 that extends substantially parallel, and coincident with, the second transverse dimension of the fixation body 34. The insertion instrument 40 defines a shaft 96 that defines a distal end 95 and an opposed proximal end 97 that is spaced from the distal end 95 along the central axis 41. The insertion instrument 40 includes a clamp 94 carried by and extending from the distal end 95 of the shaft 96, and a handle 98 carried by and extending from the proximal end 97 of the shaft 96.

The clamp 94 includes a pair of longitudinally opposed spreader arms 100 that project obliquely out from the distal end 95 of the shaft 96 at an angle with respect to the longitudinal axis 41. The free ends of the spreader arms 100 each carry an engagement member that is configured to mate with the engagement member of the fixation body 34 so as to apply an expansion force onto the arms 38. For instance, the engagement member of each of the spreader arms 100 can be provided as a tooth that is carried on an inner surface of the spreader arms 100 at a location proximate to the free end of the spreader arms 100. Each tooth 44 each projects inwardly toward the opposed tooth 44, and is configured to be received in a complementary one of the grooves 48 that extend into the arms 38 as described above.

The insertion instrument 40 further includes a carrier 106 that can be tubular and is mounted onto the shaft 96, for instance proximate to the distal end 95 of the shaft 96. The carrier 106 can carry an outer textured grip surface 108. The insertion instrument 40 further includes a bushing 104 that is fixedly disposed in the distal portion of the carrier 106. Each of the spreader arms 100 defines a fixed end 102 that is fixedly connected to the bushing 104. The bushing 104 defines an internal thread 107 which is coaxial to the longitudinal axis 41 of the insertion instrument 40. The front part of the shaft 96 is provided with an outer thread which engages the internal thread 107 of the bushing 104.

The insertion instrument 40 can be coupled to the bone fixation member by placing the engagement teeth 44 into the complementary engagement grooves 48 that extend into the arms 38, such that a surface of the teeth 44 abut the steep flank 49 (see FIG. 3) of the arms 38 as defined by the grooves 48, and the connection interface 37 is aligned with a pusher 110 that is disposed at the front end of the shaft 96, and can further be integral with the shaft 96 as desired. Accordingly, the arms 38 of the bone fixation member 30 are disposed inside the spreader arms 100 of the clamp 94. The handle 98 can then be rotated while the user manually retains the grip surface 108 of the carrier 106, which causes the shaft 96 to rotate with respect to the carrier, thereby causing the outer thread at the front part of the shaft 96 to rotate with respect to the internal thread 107 of the bushing 104. Thus, the shaft 96 and the associated pusher 110 are advanced distally relative to the carrier 106, the bushing 104, and the spreader arms 100. As the pusher 110 advances distally against the connection interface 37, the bone fixation member 30 also advances distally with respect to the spreader arms 100 and teeth 44, which thereby deflect outwardly and apply an expansion force against the arms 38 (e.g., at the steep flank 49), causing the arms 38 to flex outwardly away from each other until the width between the distal ends of the arms increases from the first initial width W1 to the second expanded width W2 that is great enough to clip the underlying bone to which the bone fixation member 30 is to be fixed, such as the lamina of a vertebra 23 (see FIG. 1). It should be appreciated that the underlying bone may be already fixed to an implant, and that the bone fixation member 30 can be clipped onto the implant. In embodiments where the bone fixation member is clipped onto the implant, it can be said that the bone fixation member is also attached to the bone. Once the lamina is disposed inside the bone receiving space 32, the insertion tool 40 can be removed from the bone fixation member 30, for instance by reversibly rotating the handle 98 until the spreader arms 100 can be easily removed from the arms 38. The bone fixation member 30 can then be fastened with respect to the fixation rod 24 in the manner described above.

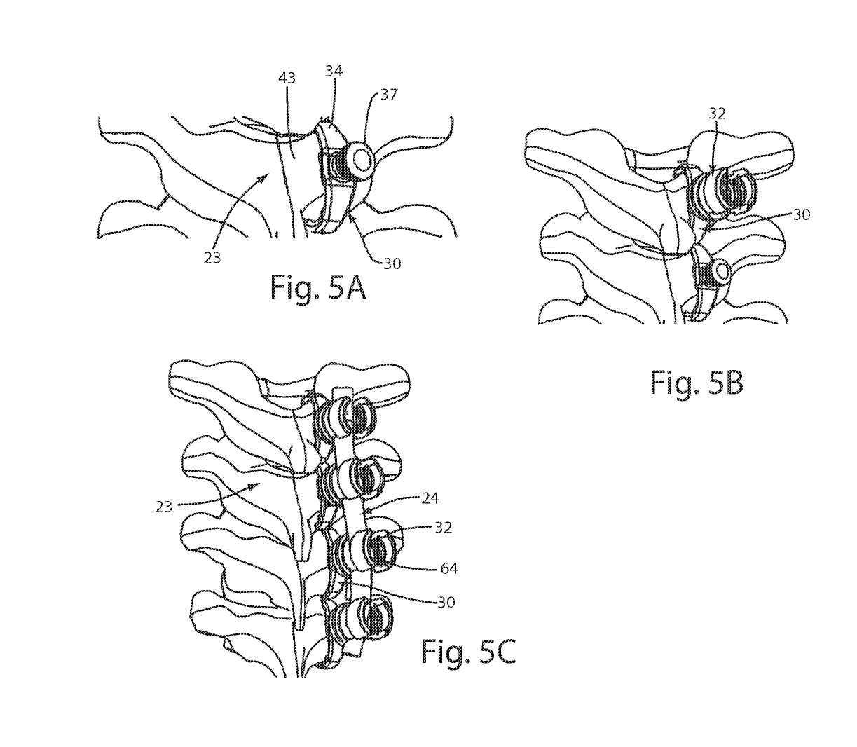

Referring now to FIG. 1 and also to FIGS. 5A-C, a method for stabilizing a part of the spinal cord, for instance in the cervical region of the spine, using the bone fixation assembly 20 including at least a first bone fixation element 22A and a second bone fixation element 22B can include at least one or more, up to all, of the following steps, alone or in combination with other steps.

1) establishing an incision each for a posterior approach to each vertebral body 23 to be treated;

2) attaching a first bone fixation member 30A of the first bone fixation element 22A to the insertion instrument 40;

3) actuating the insertion instrument so as to apply an expansion or spreading force to the first bone fixation member 30A that causes the respective arms 38 to flex outward;

4) inserting and positioning the bone fixation member 30 over the lamina 43 of a first vertebral body 23a to be treated;

5) releasing the bone fixation member 30 by reversing the shaft 96 so that the bone fixation member 30 is clipped over the lamina 24 as illustrated in FIG. 5A;

6) repeating steps 2) to 5) for a second bone fixation member 30b of a second bone fixation element 22b and second vertebral body 23b, and as many subsequent fixation members associated with each vertebral body 23 to be treated;

7) snapping a connector assembly 32 onto the connection interface 37 of each bone fixation member 30 attached to the vertebral bodies 23, as illustrated in FIG. 5B;

8) positioning a longitudinal rod 24 in the channels 64 of the connector assemblies 32 attached to the vertebral bodies 23 as illustrated in FIG. 5C;

9) mounting an insert screw 82 on each connector assembly 32;

10) fastening the insert screws 82; and

11) fastening the set screws 78 in each connector assembly 32 in order to fix the longitudinal rod 24 to the bone fixation members 30 as illustrated in FIG. 1.

Referring now to FIGS. 6-7B, the bone fixation member 30 can be constructed in accordance with an alternative embodiment. In accordance with the embodiment illustrated in FIGS. 6-7B, the connection interface 37 can include a head 52 that is configured to receive the connection assembly 32 that can be provided as a fastener 112, such as a cable tie, configured to attach the fixation rod 24 to the fixation member 30 such that the fixation rod 24 is substantially fixed with respect to movement relative to the fixation member 30. For instance, the bone fixation member 30 defines at least one aperture 114 that extends through the head 52 substantially along the lateral direction A that is substantially transverse to the plane defined by the fixation body 34. The aperture 114 is configured to receive the fastener 112 which can thus be looped around the fixation rod 24 so as to define a closable form configured to enclose an entire periphery of the fixation rod 24.

The fastener 112 can be configured as a cable tie that provides rigid fixation of the fixation rod 24 to the bone fixation member 30. In accordance with the illustrated embodiment, the tension-stable fastener 112 includes a substantially belt-shaped body 116 that can be looped and closed by a closing piece 118 having teeth that engage complementary teeth of the body 116, such that the free end of the body 116 is movable only in one direction, i.e. in a direction to make the fastener and the defined channel 64 smaller. By tightening the body 116, the bone fixation member 30 is drawn closer to the longitudinal rod 24, for instance to a degree such that the bone fixation member 30 abuts the fixation rod 24. The head 52 can define a concave abutment surface 120 that is configured to receive the longitudinal rod 24, and matches the convex outer surface of the longitudinal rod 24. The channel 16 can be elongate along a direction that is parallel to the first longitudinal axis 7 of the fixation body 34, such that the fixation rod 24 can extend along the plane defined by the fixation body 34.

In accordance with another alternative embodiment, the head 52 can widen out to admit two separate apertures 114, and the head 52 can be tipped down relative to the second transverse axis of the fixation body 34 in order to bring it closer to the longitudinal rod 24.

It should be appreciated that while various connection interfaces have been illustrated that are configured to rigidly fix a pedicle screw or hook to a fixation rod are described, for instance, in U.S. Pat. No. 6,325,802, the disclosure of which is hereby incorporated by reference as if set forth in its entirety herein.

It should be appreciated that a kit can include at least one such as a plurality of bone fixation elements 22 or components thereof, such as the bone fixation member 30 alone or in combination with a connector assembly 32, at least one such as a plurality of bone fixation rods 24, and at least one such as a plurality of insertion instruments 40.

Although the invention and its advantages have been described in detail, it should be understood that various changes, substitutions, and alterations can be made herein without departing from the spirit and scope of the invention as defined by the appended claims. Moreover, the scope of the present application is not intended to be limited to the particular embodiments of the process, machine, manufacture, and composition of matter, structure, methods and steps described in the specification. As one of ordinary skill in the art will readily appreciate from the disclosure of the present invention, processes, machines, manufacture, composition of matter, structure, methods, or steps, presently existing or later to be developed that perform substantially the same function or achieve substantially the same result as the corresponding embodiments described herein may be utilized according to the present invention.

It will be appreciated by those skilled in the art that various modifications and alterations of the invention can be made without departing from the broad scope of the appended claims. Some of these have been discussed above and others will be apparent to those skilled in the art.

1. A bone fixation element configured to attach a bone fixation rod to an underlying bone, the bone fixation element comprising a bone fixation member including 1) a fixation body including a base and a pair of spaced arms that extend from the base, and define a bone receiving space therebetween, the arms being spreadable from a first initial position to a second flexed position configured to attach the fixation body to an underlying bone, and 2) a connection interface that extends from the fixation body.

2. The bone fixation element as recited in claim 1, wherein the arms are configured to be reversibly clippable on a lamina of a vertebra.

3. The bone fixation element as recited in claim 1, wherein the bone fixation member is made from a material with a Young's modulus greater than 30 GPa.

4. The bone fixation element as recited in claim 1, wherein the bone fixation member is made from a memory metal.

5. The bone fixation element as recited in claim 1, further comprising a connector assembly that is configured to attach to both the bone fixation rod and the bone fixation member.

6. The bone fixation element as recited in claim 5, wherein the connector assembly comprises a fastener that has a closable form configured to enclose an entire periphery of the longitudinal rod.

7. The bone fixation element as recited in claim 5, wherein the connector assembly is configured to secure the bone fixation rod with respect to translational and rotational movement relative to the bone fixation member.

8. The bone fixation element as recited in claim 5, wherein the bone fixation body and the connector assembly are rigidly connected to each other.

9. The bone fixation element as recited in claim 8, wherein the connector interface is configured to receive the bone fixation rod such that the bone fixation rod extends along a direction parallel to the direction in which the arms are spaced.

10. The bone fixation element as recited in claim 5, wherein the fixation member is formed as a cable tie.

11. The bone fixation element as recited in claim 5, wherein the connection interface and the connector assembly define a releasably fixable polyaxially pivotable joint.

12. The bone fixation element as recited in claim 1, wherein the fixation body defines a substantially elliptical shape that defines a first long dimension and a second short dimension that extend in a plane defined by the arms.

13. The bone fixation element as recited in claim 1, wherein the arc-shaped element comprises more than two arms.

14. The bone fixation element as recited in claim 1, wherein the base comprises an inner surface that defines a first radius of curvature, and at least one of the arms comprises an inner surface that defines a second radius of curvature less than the first radius of curvature.

15. The bone fixation element as recited in claim 1, wherein each of the two arms defines a distal end opposite the base, such that the distal ends define a mouth to the bone receiving space having a width that expands when the arms move from their first initial position to their second flexed position.

16. The bone fixation element as recited in claim 1, wherein the each of the two arms comprises an engagement member configured to engage an insertion instrument that applies an expansion force onto the arms that causes the arms to flex away from each other.

17. The bone fixation element as recited in claim 16, wherein the engagement member comprises a groove configured to receive a complementary tooth of the insertion instrument.

18. The bone fixation element as recited in claim 1, wherein each of the arms define an inner surface which is coated with hydroxylapatite, a polymer or titanium.

19. The bone fixation element as recited in claim 18, wherein the arms are elastically spreadable from the first initial position to the second flexed position.

20. An insertion instrument configured to clip a bone fixation member having a base and a pair of opposed arms extending from the base onto an underlying bone, the insertion instrument comprising:
a clamp including an engagement member configured to engage the opposed arms of the bone fixation member; and
a shaft that carries a pusher that configured to apply a force against the base as the clamp is engaged to the opposed arms, such that as the shaft is advanced relative to the clamp, the clamp causes the opposed arms to spread apart.
